(19) United States
(12) Patent Application Publication
(10) Pub. No.: US 2010/0076193 A1
(43) Pub. Date: Mar. 25, 2010

(54) PROCESS FOR THE PREPARATION OF GEMIFLOXACIN

(76) Inventors: Chandrasekaran Ramasubbu, Tamil Nadu (IN); Suresh Ramasamy, Tamil Nadu (IN); Kiranmye Tayyala, Tamil Nadu (IN); Mahender Rao Siripragada, Tamil Nadu (IN)

Correspondence Address: LADAS & PARRY LLP, 26 WEST 61ST STREET, NEW YORK, NY, 10023, US

(21) Appl. No.: 12/513,090

(22) PCT Filed: Oct. 31, 2007
(86) PCT No.: PCT/IB07/03290

§ 371 Date: Dec. 7, 2009

(30) Foreign Application Priority Data
Oct. 31, 2006 (IN) 1995/CHE/2006

(51) Int. Cl. C07D 471/04; C07D 207/22

(52) U.S. Cl. 546/123; 548/557

(57) ABSTRACT

The present invention relates to an improved process for the preparation of Gemifloxacin mesylate of formula (V). The present invention further provides novel intermediates of formula (II) and (IV), which are useful intermediates for the preparation of Gemifloxacin mesylate of formula (V).

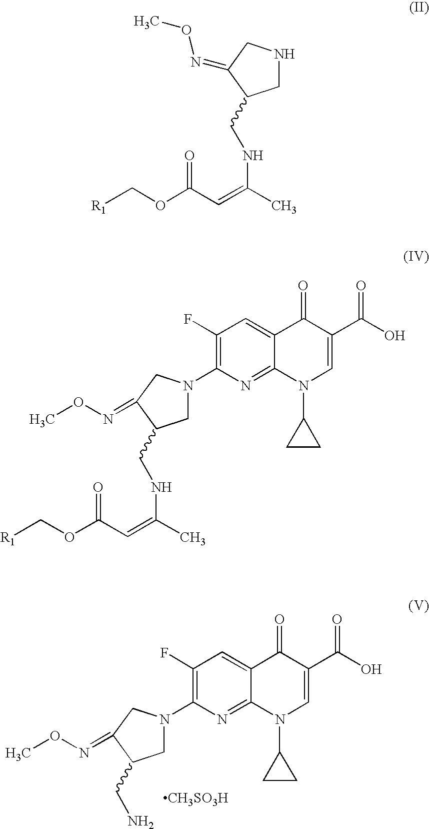

wherein R₁ is linear or branched chain alkyl group having 1-3 carbon atoms.

### FIELD OF THE INVENTION

The present invention relates to an improved process for the preparation of Gemifloxacin mesylate of formula (V). The present invention further provides novel intermediates of formula (II) and (IV), which are useful intermediates for the preparation of Gemifloxacin mesylate of formula (V).

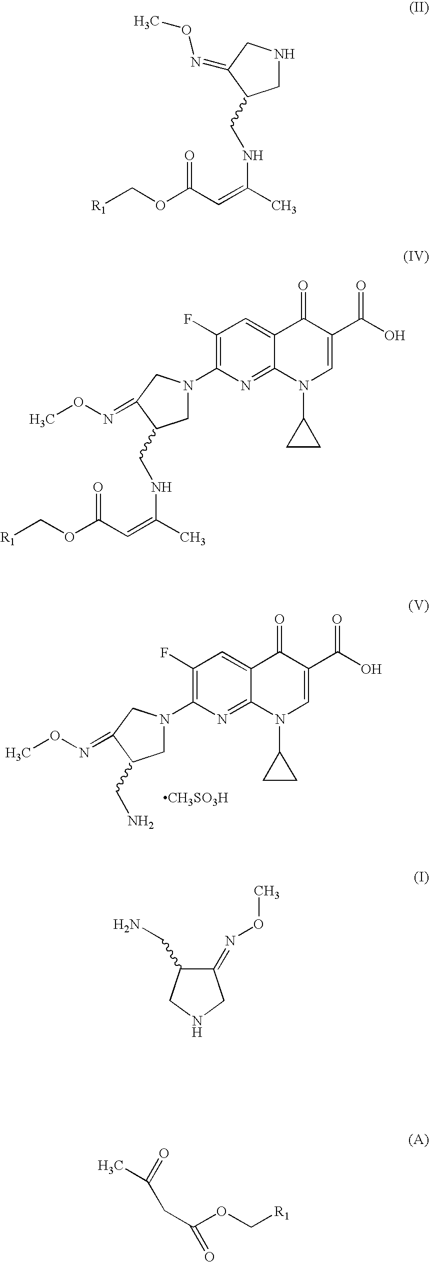

wherein R₁ is hydrogen or linear or branched chain alkyl group having 1-3 carbon atoms.

### BACKGROUND OF THE INVENTION

Gemifloxacin mesylate, which is chemically known as (±)-7-[(Z)-3-(aminomethyl)-4-(methoxyimino)pyrrolidin-1-yl]-1-cyclopropyl-6-fluoro-4-oxo-1,4 dihydro-1,8-naphthyridine-3-carboxylic acid methanesulfonate is a member of the fluoroquinolone class of antibiotics, and has the following structural formula:

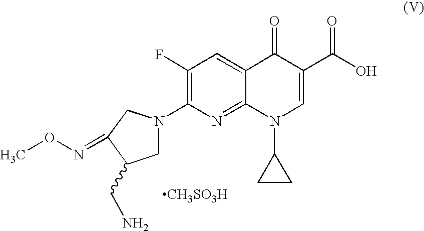

Gemifloxacin mesylate is useful as an anti-bacterial and marketed as Factive® by Oscient Pharmaceuticals. Gemifloxacin and its pharmaceutically acceptable salts are known generically from the U.S. Pat. No. 5,633,262 and specifically from the U.S. Pat. No. 5,776,944 (henceforth '944).

According to the '944, preparation of Gemifloxacin and its salt thereof is depicted below:

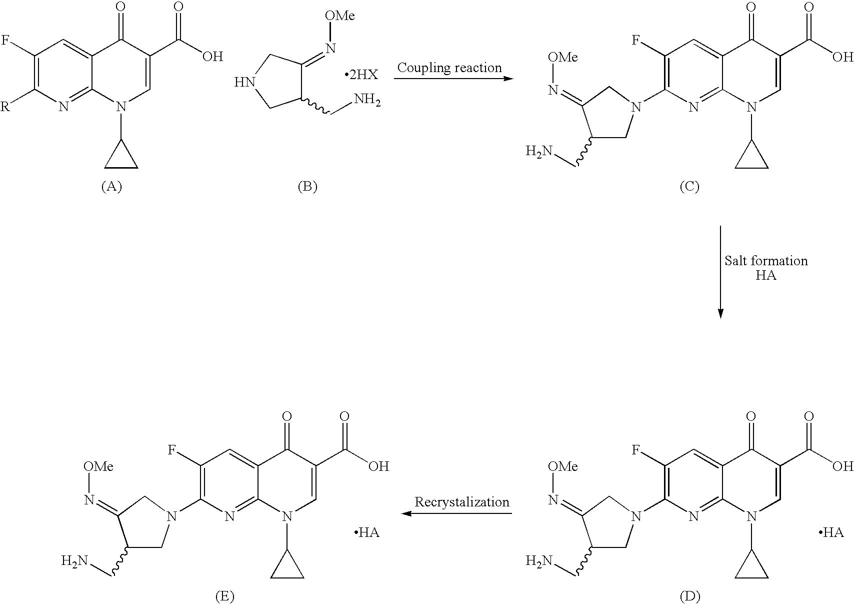

wherein, Me represents methyl, R represents Cl, F, Br, I, methanesulfonyl or paratoluenesulfonyl, HX represents hydrochloric acid, hydrobromic acid, hydroiodic acid, trifluoroacetic acid, methanesulfonic acid, para toluenesulfonic acid, or sulfuric acid, HA is an organic acid or an inorganic acid.

As shown in the above reaction scheme, the compound (E) is prepared through a three-step reaction process, i.e., coupling reaction, salt formation and re-crystallization. The reason why the three-step reaction process is carried out is because the compound (VI) as dimeric compound is formed in an amount of approximately 6-12% as process impurity in the coupling reaction and the compound (VI) is remained in the compound (C) in an amount of approximately 0.3 to 1.0%. To reduce this impurity in the coupling reaction to 0.1% or less, the second step, that is a salt formation process, had to be carried out. Finally, the organic solvent used in the salt formation process had to be removed from the step of re-crystallization.

Through the three-step process, an acid salt of Gemifloxacin as a raw medicine having high purity was prepared in about 65% of total yield. Since the resulting impurity (VI) from the coupling reaction of the above process was difficult to be avoided, the salt formation and re-crystallization steps for removing the impurity had to be carried out.

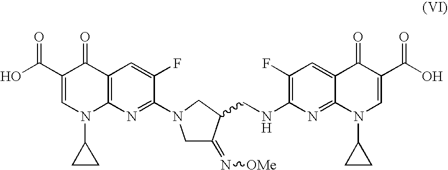

U.S. Pat. No. 5,869,670 which is a divisional patent of the '944, claims the process of preparation of Gemifloxacin or its isomer, methanesulphonate and hydrate of the methanesulphonate, which comprises reacting a quinolone, with a protected pyrrolidine oxime in the presence of a base and then removing the amino-protecting group (formyl, acetyl, trifluoroacetyl, benzoyl, para-nitrobenzoyl, para-toluenesulfonyl, methoxycarbonyl, ethoxycarbonyl, t-butoxycarbonyl, benzyloxycarbonyl, para-methoxybenzyloxycarbonyl, trichloroethoxycarbonyl, benzyl, para-methoxybenzyl, trityl and tetrahydropyranyl) from the resulting compound.

US 2005/0148622 A1 claims a two step process for preparing acid salt of Gemifloxacin comprising the steps of: (a) adding a compound of formula 5 (which may be selected form benzaldehyde, 2-chlorobenzaldehyde, 2-hydroxy-benzaldehyde, 4-methoxybenzaldehyde and 1-naphthaldehyde) to napthyridine carboxylic acid of formula (2) and 3-aminomethyl-4-methoxyiminopyrrolidine salt of formula (3) in water, an organic solvent or a mixed solvent thereof in the presence of an organic base (which may be selected from triethylamine, trimethylamine, diisopropylethylamine, 1,8-diazabicyclo[5.4.0]undec-7-ene, and 1,5-diazabicyclo[4.3.0]non-5-one) to carry out a coupling reaction, and (b) adding an acid of formula HA to the resulting compound of formula (4) in water, an organic solvent or a mixed solvent thereof to carry out deprotection and salt formation reaction simultaneously; wherein, R represents Cl, F, Br, I, methansulfonyl or para toluene sulfonyl, Me represents methyl, HX represents hydrochloric acid, hydrobromic acid, hydroiodic acid, trifluoroacetic acid, methanesulfonic acid, paratoluenesulfonic acid or sulphuric acid. R₁ and R₂ independently of each other represent hydrogen, a straight or branched, saturated or unsaturated C₁-C₆ alkyl group, a saturated or unsaturated C₃-C₆ cycloalkyl group, or an aromatic group which is unsubstituted or substituted by C₁-C₆ alkyl, C₁-C₆ alkoxy, hydroxy, cyano or halogen, or R₁ and R₂ together with a carbonyl group to which they are bonded form a ring and HA is an organic acid or an inorganic acid. The organic solvent used in step (a) is acetonitrile and in step (b) is isopropanol or tetrahydrofuran.

The reaction sequence is depicted below:

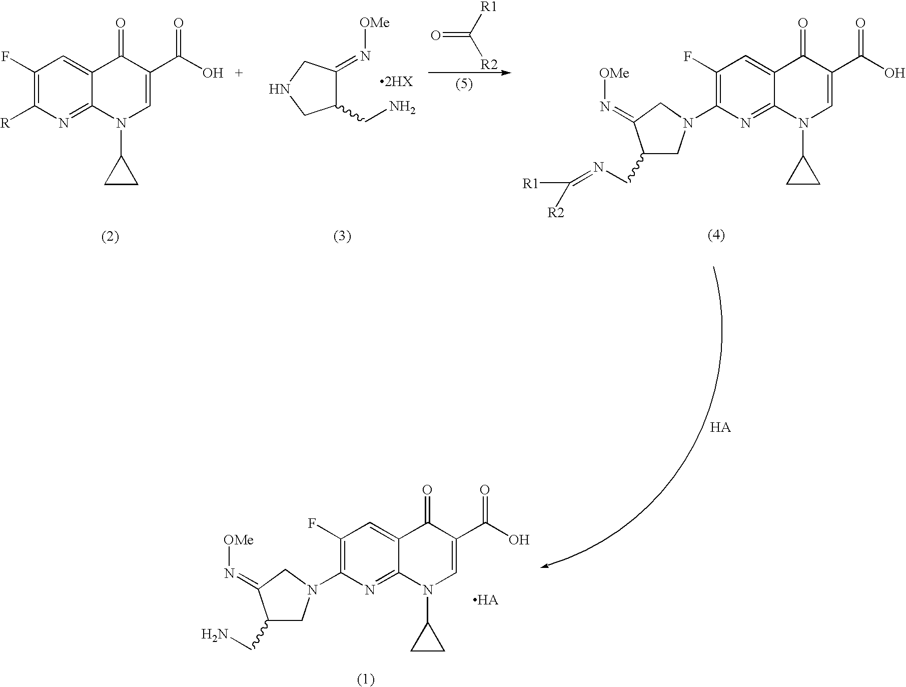

With reference to the above-discussed procedures none of the prior art references disclosed or claimed the protection of amino group of formula (I) by reacting a compound of formula A to produce novel intermediates of formula (II) and (IV) which are useful for the preparation of Gemifloxacin mesylate of formula (V).

We focused our research to develop an industrially useful and efficient process for the preparation of the compound of formula (V) in substantially good yield and high chemical purity.

### OBJECTIVES OF THE INVENTION

The main objective of the present invention is to provide a process for the preparation of compound of formula (V) in higher yield and greater chemical purity.

Another objective of the present invention is to provide novel intermediates of a compound of formula (II) and (IV), which are useful in the preparation of Gemifloxacin mesylate of formula (V).

Yet another objective of the present invention is to provide a process for the preparation of compound of formula (V), which would be much simpler, more economical and easy to implement on commercial scale.

### SUMMARY OF THE INVENTION

Accordingly, the present invention provides an improved process for the preparation of Gemifloxacin mesylate of formula (V), which comprises the steps of:

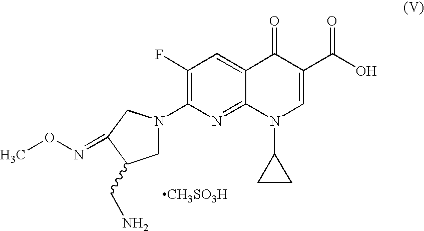

(i) protecting the amino group of a compound of formula (I) or salts thereof by reacting compound A in an organic solvent or aqueous organic solvent in the presence of an organic base to get an intermediate of formula (II);

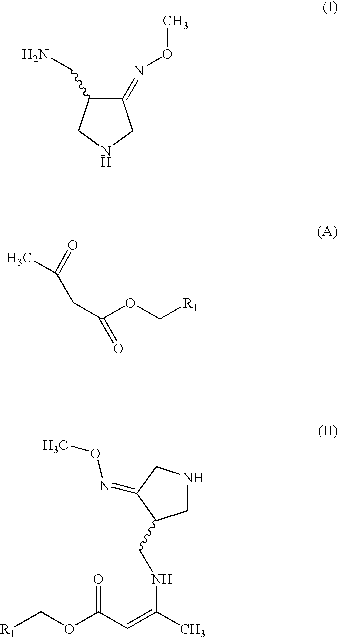

wherein R₁ is hydrogen or branched chain alkyl group having 1-3 carbon atoms such as methyl, ethyl, propyl and the like.
(ii) condensing the intermediate of formula (II) with a compound of formula (III) to get an intermediate of formula (IV);

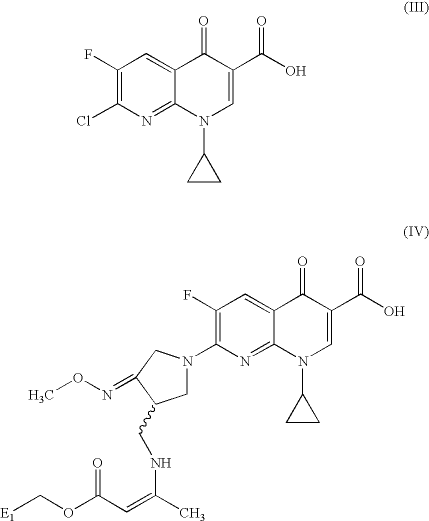

(iii) purifying the intermediate of formula (IV) and;
(iv) deprotecting the intermediate of formula (IV) using methanesulfonic acid in an organic solvent or aqueous organic solvent to produce the Gemifloxacin mesylate of formula (V).
   The process is shown in the scheme given below:

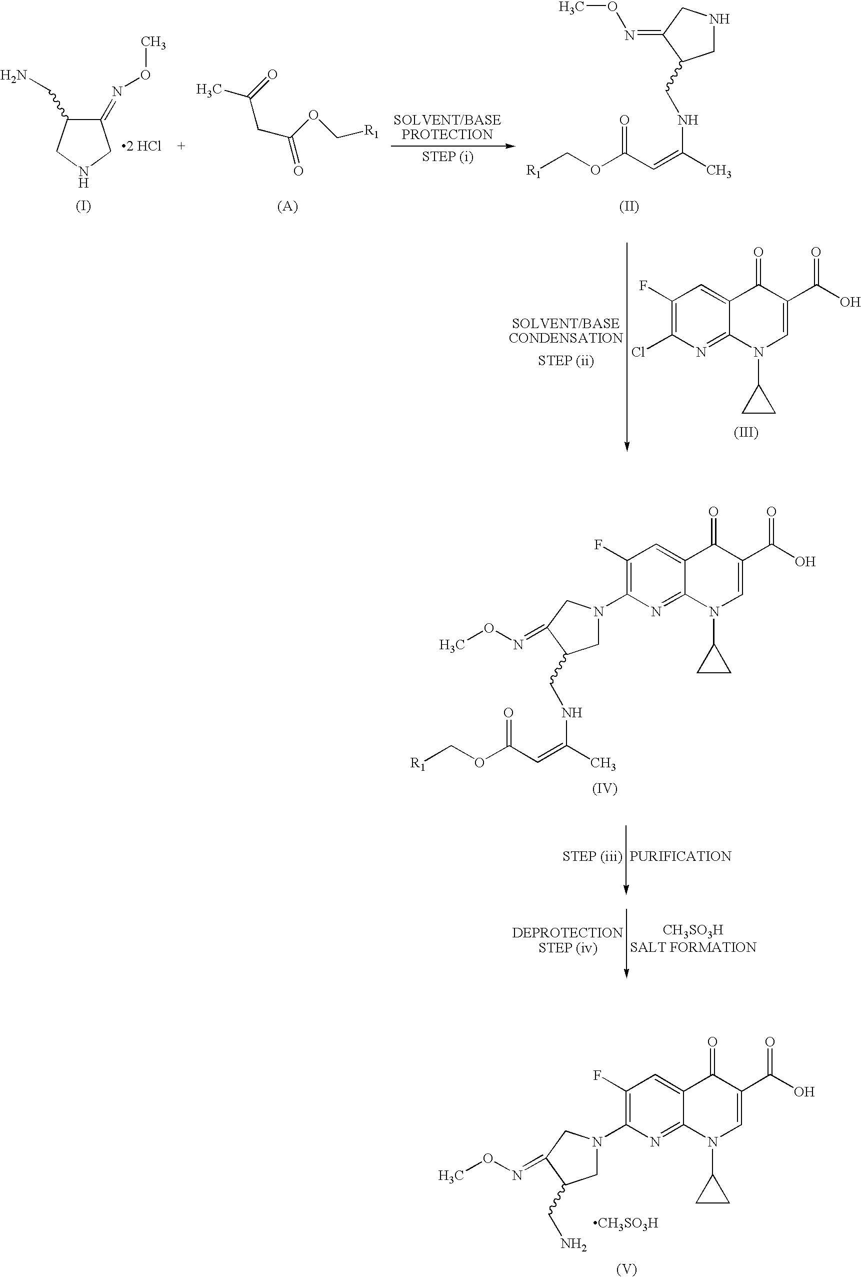

### DESCRIPTION OF THE INVENTION

In an embodiment of the present invention, the compound of formula (A) in step (i) is preferably selected from methyl acetoacetate, ethyl acetoacetate and the like.

In another embodiment of the present invention, the organic solvent used in step (i) and step (ii) is selected from methanol, ethanol, n-propyl alcohol, isopropyl alcohol, n-butanol, isobutanol, tertiary butanol, acetonitrile and the like or mixtures thereof; most preferably methanol.

In another embodiment of the present invention, the organic base used in step (i) and step (ii) is selected from triethylamine, diethylamine, pyridine, N,N-diethyl methylamine, N,N-diethylaniline, N,N-diethylethylenediamine, N,N-diisopropylethylamine, N,N-dimethylaminopyridine, N,N-diisopropylethylamine, N-methylmorpholine, N-methylpyrrolidine, 2,6-di-tert-butyl-4-methylpyridine and the like; most preferably triethylamine.

In another embodiment of the present invention, the step (i) and step (ii) are preferably performed at a temperature in the range of (−) 10° C. to reflux temperature of the solvent used.

In another embodiment of the present invention, the step (i) and step (ii) are preferably performed as a single pot reaction.

In another embodiment of the present invention, the organic solvent used in step (iii) is selected from chloroform, dichloromethane, carbon tetrachloride, methanol, ethanol, n-propyl alcohol, isopropyl alcohol, n-butanol, isobutanol, tertiary butanol and the like or mixture thereof; most preferably chloroform and isopropyl alcohol.

In another embodiment of the present invention the compound of formula (IV) is subjected to purification steps. The purification steps lead to get the final compound in highly pure form. The purification step (iii) involves subjecting the compound of formula (IV) for carbon treatment and/or re-crystallization of compound of formula (IV) in an organic solvent described above; most preferably re-crystallization is performed in chloroform and isopropyl alcohol.

In the present invention the reagents used for the protection of amino group of formula (I) is very cheap and commercially available.

In another embodiment of the present invention, the step (iii) is preferably performed at a temperature in the range of 20° C. to reflux temperature of the solvent system.

In yet another embodiment of the present invention, the organic solvent used in step (iv) is selected from methanol, ethanol, n-propyl alcohol, isopropyl alcohol, n-butanol, isobutanol, tertiary butanol, chloroform, dichloromethane, carbon tetrachloride and the like or mixtures thereof.

In still another embodiment of the step (iv) is preferably performed at a temperature in the range of 20° C. to reflux temperature and most preferably at a reflux temperature of the solvent system.

The compound of formula (IV) according to the present invention can be further used to form a pharmaceutically acceptable non-toxic salt of Gemifloxacin. Such salt includes a salt with inorganic acids such as hydrochloric acid, hydrobromic acid, phosphoric acid, sulfuric acid, etc., a salt with organic carboxylic acids such as acetic acid, trifluoroacetic acid, citric acid, maleic acid, oxalic acid, succinic acid, benzoic acid, tartaric acid, fumaric acid, mandelic acid, ascorbic acid or malic acid or with sulfonic acids such as methanesulfonic acid, para-toluenesulfonic acid, etc., and a salt with other acids which are generally known and conventionally used in the technical field of quinolone-based compounds. These acid-addition salts can be prepared according to a conventional conversion method.

In present invention the starting materials are prepared according to the literature available in the prior art.

The present invention is illustrated with the following examples, which should not be construed for limiting the scope of the invention.

### Example 1

### Preparation of 6-Fluoro-7-[3-({[(1Z)-3-ethoxy-1-methyl-3-oxoprop-1-en-1-yl]amino}methyl)-4-(methoxyimino)-pyrrolidin-1-yl]-1-cyclopropyl-4-oxo-1,4-dihydro-1,8-naphthyridine-3-carboxylic acid (IV; R₁=methyl)

4-(Aminomethyl)pyrrolidin-3-one O-methyloxime hydrochloride (25 gm) was dissolved in methanol (375 mL) and triethylamine (49 mL) added drop wise at 25 to 30° C. To the clear solution ethyl acetoacetate (18 gm) was added dropwise. The reaction mixture was stirred for 30 to 45 mins to get ethyl (2Z)-3-({[(4Z)-4-(methoxyimino)pyrrolidin-3-yl]methyl}amino)but-2-enoate and then 7-chloro-1-cyclopropyl-6-fluoro-4-oxo-1,4-dihydro-1,8-naphthyridin-3-carboxylic acid (27.8 gm) was added. The reaction mass was heated to reflux temperature and maintained till the completion of reaction. The reaction mass was cooled, filtered washed with methanol and dried under vacuum to get 42 gm of 6-Fluoro-7-[3-({[(1Z)-3-ethoxy-1-methyl-3-oxoprop-1-en-1-yl]amino}methyl)-4-(methoxyimino)-pyrrolidin-1-yl]-1-cyclopropyl-4-oxo-1,4-dihydro-1,8-naphthyridine-3-carboxylic acid (Yield: 85.05% and purity by HPLC is >94%.)

### Example 2

### Purification of 6-Fluoro-7-[3-({[(1Z)-3-ethoxy-1-methyl-3-oxoprop-1-en-1-yl]amino}methyl)-4-(methoxyimino)-pyrrolidin-1-yl]-1-cyclopropyl-4-oxo-1,4-dihydro-1,8-naphthyridine-3-carboxylic acid

6-Fluoro-7-[3-({[(1Z)-3-ethoxy-1-methyl-3-oxoprop-1-en-1-yl]amino}methyl)-4-(methoxyimino)-pyrrolidin-1-yl]-1-cyclopropyl-4-oxo-1,4-dihydro-1,8-naphthyridine-3-carboxylic acid (38 gm) was dissolved in chloroform (342 mL) at 25° C. to 30° C. and charcoalized (30 gm). The reaction mass was filtered and filtrate was concentrated by distilling out chloroform and the resultant residual mass cooled. Isopropyl alcohol (152 mL) was added slowly under rigorous stirring. The product thus obtained was filtered, washed with isopropyl alcohol and the same was re-crystallized in isopropyl alcohol (133 mL)-chloroform mixture (152 mL). The product thus obtained was filtered, washed and dried at 50 to 55° C. under vacuum to get pure 24 gm of 6-Fluoro-7-[3-({[(1Z)-3-ethoxy-1-methyl-3-oxoprop-yl]amino}methyl)-4-(methoxyimino)-pyrrolidin-1-yl]-1-cyclopropyl-4-oxo-1,4-dihydro-1,8-naphthyridine-3-carboxylic acid (Purity by HPLC is >98%).

Melting point: 219-223° C.

Mass (m/e): 502 (M+1)

¹H-NMR (CDCl₃): δ 14.98 (1H, s), 8.8 (1H, t), 8.7 (1H, s), 8.0 (1H, d), 4.6 (2H, s), 4.5 (1H, s), 4.04 (1H, m), 4.03 (2H, q), 3.9 (3H, s, 1H, m), 3.65 (2H, m), 3.4 (1H, m), 3.3 (1H, m), 1.9 (3H, s), 1.3 (2H, d), 1.2 (3H, t), 1.0 (2H, d),

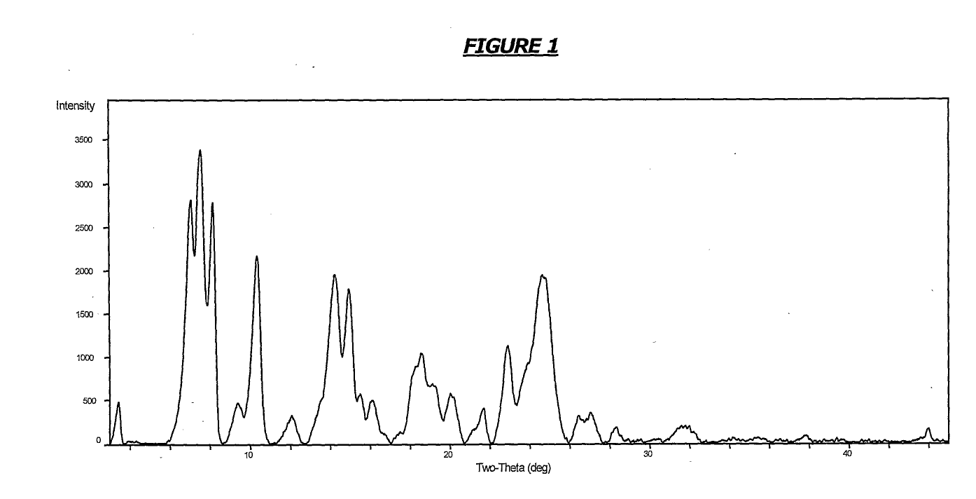

X-ray powder diffraction pattern has 2 theta angles: 3.42, 7.06, 7.56, 8.16, 9.38, 10.36, 12.06, 14.26, 14.94, 15.50, 16.08, 18.54, 19.15, 20.05, 21.68, 22.90, 24.76, 26.42, 27.04, 28.33, 31.78, 37.84, 43.98 (As per the FIG. 1).

Differential Scanning Colorimetric (DSC) thermogram exhibiting a significant endo peak at 223.21° C.

### Example 3

### Preparation of 7-[(4Z)-3-(Aminomethyl)-4-(methoxyimino)pyrrolidin-1-yl]-1-cyclopropyl-6-fluoro-4-oxo-1,4-dihydro-1,8-naphthyridine-3-carboxylic acid methane sulphonate (V)

6-Fluoro-7-[3-({[(1Z)-3-ethoxy-1-methyl-3-oxoprop-1-en-1-yl]amino}methyl)-4-(methoxyimino)-pyrrolidin-1-yl]-1-cyclopropyl-4-oxo-1,4-dihydro-1,8-naphthyridine-3-carboxylic acid (19 gm) was dissolved in methylene dichloride (300 mL) and ethanol (38 mL) at 25° C. to 30° C. to get a solution. To this solution methanesulphonic acid in methylene dichloride was added and stirred till the completion of reaction. Solid material obtained was filtered, washed with ethanol and dried under vacuum to give 18 gm of 7-[(4Z)-3-(aminomethyl)-4-(methoxyimino)pyrrolidin-1-yl]-1-cyclopropyl-6-fluoro-4-oxo-1,4-dihydro-1,8-naphthyridine-3-carboxylic acid mesylate. (Purity by HPLC>99.5%).

Melting point: 195° C.

Moisture content: 0.54%

¹H-NMR (DMSO-d6): δ15.26 (1H, S), δ 8.59 (1H, S), δ 8.04 (1H, d), δ 7.95 (2H, Broad signal), δ 4.58 (2H, S), δ 3.90 (3H, S), δ 3.83& δ 4.38 (2H, Two multiplets), δ 3.71 (1H, m),

δ 3.41 (1H, m), δ 3.18 (2H, m), δ 2.33 (3H, S), δ 1.22 (2H, Two broad singlets),

δ 1.08 (2H, m)

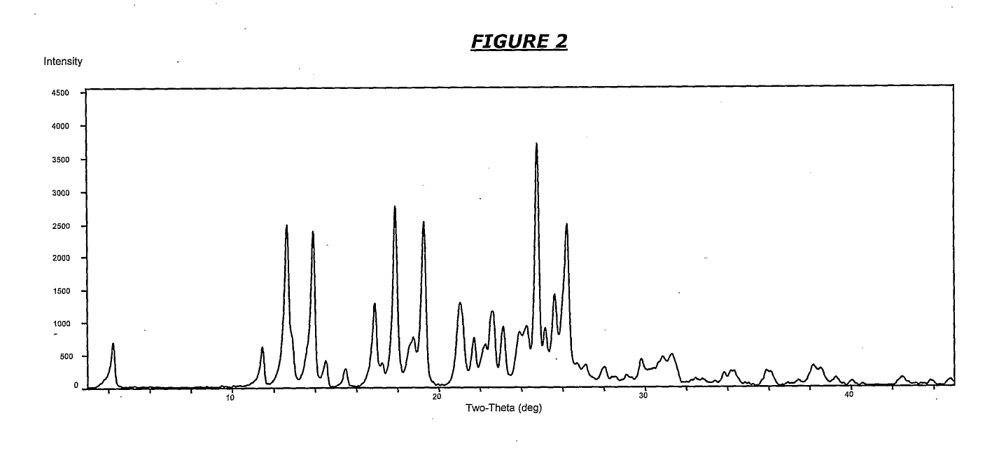

Powder method of X Ray Diffraction (Characteristic peak) 2θ=4.23, 12.66, 13.92, 16.90, 17.90, 19.28, 24.78, 26.22. (As per the FIG. 2).

### Example 4

### Preparation of 7-[(4Z)-3-(Aminomethyl)-4-(methoxyimino)pyrrolidin-1-yl]-1-cyclopropyl-6-fluoro-4-oxo-1,4-dihydro-1,8-naphthyridine-3-carboxylic acid methane sulphonate sesquihydrate (V)

6-Fluoro-7-[3-({[(1Z)-3-ethoxy-1-methyl-3-oxoprop-1-en-1-yl]amino}methyl)-4-(methoxyimino)-pyrrolidin-1-yl]-1-cyclopropyl-4-oxo-1,4-dihydro-1,8-naphthyridine-3-carboxylic acid (25 gm) was suspended in isopropyl alcohol (150 mL) and water (50 mL). The slurry mass was stirred and maintained at 45 to 50° C., methanesulphonic acid (4.6 gm) was added drop-wise and stirred well. The clear solution was cooled to 25 to 30° C. and maintained for 60 to 90 mins. The reaction mass was further cooled to 0 to 5° C., stirred for 3 hrs, filtered and the wet material was washed with isopropyl alcohol (50 mL). The wet material was slurred in isopropyl alcohol (100 mL) and water (50 mL) under stirring and slurry mass was heated to 45 to 50° C. to form a clear solution. The clear solution was cooled to 25 to 30° C. and stirred for 18 to 22 hrs at the same temperature. After stirring the slurry was cooled to 0 to 5° C. and maintained for about 2 hrs. The product thus formed was filtered, washed with isopropyl alcohol and dried under vacuum to get 18.5 gm of 7-[(4Z)-3-(aminoethyl)-4-(methoxyimino)pyrrolidin-1-yl]-1-cyclopropyl-6-fluoro-4-oxo-1,4-dihydro-1,8-naphthyridine-3-carboxylicacid mesylate sesquihydrate. (Yield: 72.4% and Purity by HPLC 99.85% E-isomer: 0.11%, and other impurities: Not detected).

1. A process for the preparation of Gemifloxacin mesylate of formula (V), which comprises the steps of: 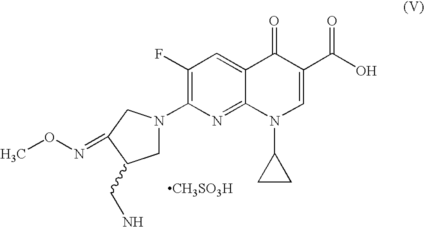
protecting the amino group of a compound of formula (I) or salts thereof by reacting compound A in an organic solvent or aqueous organic solvent in the presence of an organic base to get an intermediate of formula (II); 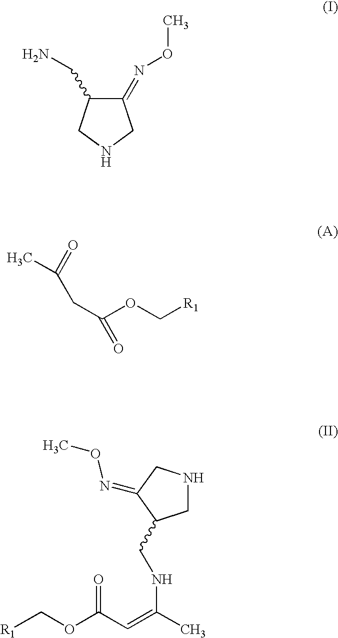
wherein R₁ is hydrogen or linear or branched chain alkyl group having 1-3 carbon atoms.
(ii) condensing the intermediate of formula (II) with a compound of formula (III) to get an intermediate of formula (IV); 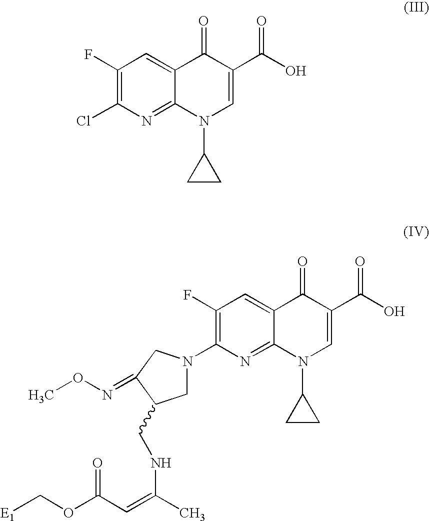
(iii) purifying the intermediate of formula (IV) and;
(iv) deprotecting the intermediate of formula (IV) using methanesulfonic acid in an organic solvent or aqueous organic solvent to produce the Gemifloxacin mesylate of formula (V).

2. A process according to claim 1, wherein the organic solvent used in step (i) and step (ii) is selected from methanol, ethanol, n-propyl alcohol, isopropyl alcohol, n-butanol, isobutanol, tertiary butanol, acetonitrile or mixtures thereof; most preferably methanol.

3. A process according to claim 1, wherein the organic base used in step (i) and step (ii) is selected from triethylamine, diethylamine, pyridine, N,N-diethyl methylamine, N,N-diethyl aniline, N,N-diethylethylenediamine, N,N-diisopropyl-ethylamine, N,N-dimethylaminopyridine, N,N-diisopropylethylamine, N-methylmorpholine, N-methylpyrrolidine, 2,6-di-tertbutyl-4-methylpyridine; most preferably triethylamine.

4. A process according to claim 1, wherein the organic solvent used in step (iii) is selected from chloroform, dichloromethane, carbon tetrachloride, methanol, ethanol, n-propyl alcohol, isopropyl alcohol, n-butanol, isobutanol, tertiary butanol or mixture thereof.

5. A process according to claim 1, wherein the organic solvent used in step (iv) is selected from chloroform, dichloromethane, carbon tetrachloride, methanol, ethanol, n-propyl alcohol, isopropyl alcohol, n-butanol, isobutanol, tertiary butanol or mixture thereof.

6. A process according to claim 1, wherein the process is performed at a temperature in the range of −10° C. to reflux temperature of the solvent system.

7. A process according to claim 1, wherein the purification step (iii) is performed by carbon treatment and/or re-crystallization in a mixture of solvent (s) selected from chloroform, dichloromethane, carbon tetrachloride, methanol, ethanol, n-propyl alcohol, isopropyl alcohol, n-butanol, isobutanol, tertiary butanol or mixture thereof.

8. An intermediate represented by the following formula (II), 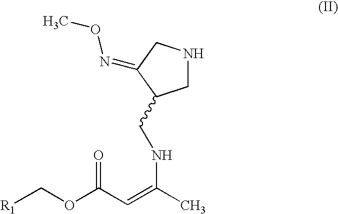
wherein R₁ is hydrogen or linear or branched chain alkyl group having 1-3 carbon atoms.

9. An intermediate represented by the following formula (IV), for preparing Gemifloxacin mesylate of compound formula (V): 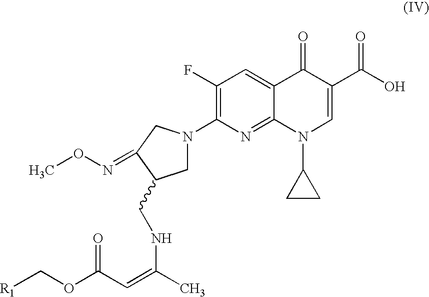
wherein R₁ is hydrogen or linear or branched chain alkyl group having 1-3 carbon atoms.

10. A method for production of Gemifloxacin or a pharmaceutically acceptable salt thereof which comprises removal of a protection group from an intermediate of formula (IV) as claimed in claim 9.

11. A crystalline solid intermediate 6-Fluoro-7-[3-({[(1Z)-3-ethoxy-1-methyl-3-oxoprop-1-en-1-yl]amino}methyl)-4 (methoxyimino)-pyrrolidin-1-yl]-1-cyclopropyl-4-oxo-1,4-dihydro-1,8-naphthyridine-3-carboxylic acid, characterized by an x-ray powder diffraction pattern expressed in terms of 2θ at about 3.42, 7.06, 7.56, 8.16, 9.38, 10.36, 12.06, 14.26, 14.94, 15.50, 16.08, 18.54, 19.15, 20.05, 21.68, 22.90, 24.76, 26.42, 27.04, 28.33, 31.78, 37.84, 43.98.

12. Use of an intermediate as claimed in claim 10 is in the preparation of Gemifloxacin or its pharmaceutically acceptable salt.

13. An anhydrate form of Gemifloxacin mesylate (Form A) characterized by an x-ray powder diffraction pattern expressed in terms of 2θ at about 4.23, 12.66, 13.92, 16.90, 17.90, 19.28, 24.78, 26.22.

14. A process according to claim 1, wherein the organic solvent used in step (iii) is selected from chloroform and isopropyl alcohol.
